(19) United States
(12) Patent Application Publication
(10) Pub. No.: US 2022/0280490 A1
(43) Pub. Date: Sep. 8, 2022

(54) TREATMENT OF FRAGILE X SYNDROME

(71) Applicant: HEALX LTD, Cambridge (GB)

(72) Inventor: David BROWN, Cambridge (GB)

(21) Appl. No.: 17/634,905

(22) PCT Filed: Aug. 14, 2020
(86) PCT No.: PCT/GB2020/051949

§ 371 Date: Feb. 11, 2022

(30) Foreign Application Priority Data
Aug. 14, 2019 (GB) 1911603.7

(51) Int. Cl. A61K 31/437

(52) U.S. Cl. CPC A61K 31/437

(57) ABSTRACT

The present invention relates to a composition comprising ibudilast, or a pharmaceutically acceptable salt thereof, for use in the treatment of Fragile X syndrome, wherein the composition does not comprise sulindac, or a pharmaceutically acceptable salt thereof, or bumetanide, or a pharmaceutically acceptable salt thereof.

### FIELD OF THE INVENTION

This invention relates to the use of ibudilast in the treatment of Fragile X syndrome (FXS).

### BACKGROUND OF THE INVENTION

Fragile X syndrome, often referred to as Fragile X, is the most common inherited cause of intellectual impairment and the most common monogenic cause of autism. It affects around 1 in 4000 males and 1 in 6000 females worldwide.

There are a wide range of characteristics associated with Fragile X, and typically males are more affected than females. One of the major characteristics associated with Fragile X syndrome is intellectual impairment, such as difficulties with cognitive, executive and language performance. Individuals with Fragile X syndrome typically have social anxiety characterised by social, emotional and communication difficulties related to extreme shyness, poor eye contact and challenges forming peer relationships. Fragile X syndrome is also associated with hyperactivity and disruptive behaviour, such as short attention span, distractibility, impulsiveness, restlessness, over-activity and sensory problems. Furthermore, individuals with Fragile X syndrome often suffer from seizures.

Fragile X syndrome arises from a mutation in a single gene called Fragile X Mental Retardation Gene 1 (FMR1). The 5′ UTR of FMR1 contains a CGG trinucleotide repeat that is polymorphic in the population. Once the repeats exceed 200 in number, methylation of the promoter is triggered, and this in turn causes the lack of expression of the gene and translation of its encoded protein, the Fragile X Mental Retardation Protein (FMRP). FMRP is an RNA-binding protein involved in different steps of mRNA metabolism, such as translational control (in soma and dendritic spines) and RNA transport.

At present, there is no effective therapy to treat Fragile X syndrome. However, there have been considerable efforts to identify pharmacological targets to treat this disorder. In particular, Fragile X syndrome has been a frequent target of repurposing efforts as well as repositioning of drugs in development. Many different standards and methods have been applied to this task. In many cases, repurposing candidates have been identified based primarily on clinical pattern matching, while in others basic disease mechanisms have been studied extensively to identify therapeutic targets, followed by thorough preclinical validation.

Overall, efforts to treat Fragile X syndrome have led to some exciting possibilities, but no definitive successes, despite much effort. This has highlighted the need for new therapies.

Ibudilast is a phosphodiesterase inhibitor and is used as an anti-inflammatory. It is used in the treatment of asthma, stroke and multiple sclerosis.

Ibudilast has the systematic name 2-methyl-1-(2-propan-2-ylpyrazolo[1,5-a]pyridin-3-yl)propan-1-one.

### SUMMARY OF THE INVENTION

The present invention is a composition comprising ibudilast, or a pharmaceutically acceptable salt thereof, for use in the treatment of Fragile X syndrome, wherein the composition does not comprise sulindac, or a pharmaceutically acceptable salt thereof, or bumetanide, or a pharmaceutically acceptable salt thereof. As will be evident from the in vivo data presented below, ibudilast is effective in treating Fragile X syndrome.

A first aspect of the invention is a composition comprising ibudilast, or a pharmaceutically acceptable salt thereof, for use in the treatment of Fragile X syndrome, wherein the composition does not comprise sulindac, or a pharmaceutically acceptable salt thereof, or bumetanide, or a pharmaceutically acceptable salt thereof.

A second aspect of the invention is use of ibudilast, or a pharmaceutically acceptable salt thereof, for the manufacture of a medicament for use in the treatment of Fragile X syndrome, wherein the medicament does not comprise sulindac, or a pharmaceutically acceptable salt thereof, or bumetanide, or a pharmaceutically acceptable salt thereof.

A third aspect of the invention is provided a method of treating Fragile X syndrome comprising administering the patient with a composition comprising ibudilast or a pharmaceutically acceptable salt thereof, wherein the composition does not comprise sulindac, or a pharmaceutically acceptable salt thereof, or bumetanide, or a pharmaceutically acceptable salt thereof.

### DESCRIPTION OF THE FIGURES

FIG. 1 shows the results from ibudilast in vivo testing.

### DETAILED DESCRIPTION

As Fragile X is a syndrome, there are a number of different manifestations and symptoms in patients. These include; intellectual impairment, such as difficulties with cognitive, executive and language performance, short-term memory, executive function, visual memory and visual-spatial relationships;

autism; social anxiety (i.e. difficulties in social interaction) such as poor eye contact, gaze aversion, prolonged time to commence social interaction, and challenges forming peer relationships; hyperactivity and repetitive behaviour, including very short attention spans, hypersensitivity to visual, auditory, tactile, and olfactory stimuli, distractibility, impulsiveness, restlessness and over-activity; disruptive behaviour, including fluctuating mood, irritability, self-injury and aggression; obsessive compulsive disorder (OCD); ophthalmologic problems, such as strabismus; seizures; difficulties with working memory, which involves the temporary storage of information while processing the same or other information; difficulties with phonological memory (or verbal working memory); and Fragile X-related primary ovarian insufficiency (FXPOI).

In the present invention, and as demonstrated by the below in vivo data, ibudilast is used to treat one or more of the above symptoms, and is therefore an effective treatment of Fragile X syndrome. Preferably, ibudilast is used for the treatment of Fragile X syndrome, wherein the patient is exhibiting typical symptoms of the syndrome including social anxiety, hyperactivity, memory loss and/or disruptive behaviour. More preferably, ibudilast is used for the treatment of Fragile X syndrome, wherein the patient is exhibiting hyperactivity, memory loss and/or disruptive behaviour.

The term “hyperactivity” has its normal meaning in the art. Hyperactivity may include having very short attention spans, hypersensitivity to visual, auditory, tactile, and olfactory stimuli, distractibility, impulsiveness, restlessness and/or over-activity.

The term “social anxiety” has its normal meaning in the art. It may also be termed as difficulties in social interaction or low sociability. Social anxiety may include having poor eye contact, gaze aversion, prolonged time to commence social interaction, social avoidance or withdrawal and challenges forming peer relationships.

The term “memory loss” has its normal meaning in the art. It may also be called memory impairment. It refers to an inability to retain information either short-term or long-term. It may include difficulties with cognitive, executive and language performance, executive function and visual memory. It may also include difficulties with working memory, also called short-term memory (i.e. the temporary storage of information while processing the same or other information) and difficulties with phonological memory (or verbal working memory). The term “disruptive behaviour” has its normal meaning in the art. It may also include repetitive behaviour. It may also include fluctuating mood, irritability, self-injury and aggression.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

The present invention is directed to a composition comprising ibudilast, or a pharmaceutically acceptable salt thereof, for use in the treatment of Fragile X syndrome, wherein the composition does not comprise sulindac, or a pharmaceutically acceptable salt thereof, or bumetanide, or a pharmaceutically acceptable salt thereof.

The composition according to the present invention does not comprise sulindac, or a pharmaceutically acceptable salt thereof. Sulindac is a non-steroidal anti-inflammatory drug (NSAID). Sulindac is used in the treatment of acute and chronic inflammatory conditions, such as arthritis, shoulder bursitis and tendonitis, as it exhibits anti-inflammatory, analgesic and antipyretic activities. The composition according to the present invention does not contain any sulindac i.e. 0 wt % sulindac.

The composition according to the present invention does not comprise a substance capable of modulating intracellular calcium concentration such as a retinoic acid—related orphan receptor—alpha (RORA) agonist, a calcium channel inhibitor or an inhibitor of the solute carrier family 12 member 1, solute carrier family 12 member 1, 2, 4 or solute carrier family 12 member 1, 2, 4, 5, dihydropyridines, phenylakylamines, benzothiazepines, indolazines, aminoglycosides, and 4-substituted derivatives of sulfamoylbenzoic acid (e.g. bumetanide, AqB007, AqB011, PF-2178, BUM13, BUM5 and bumepamine).

Specifically, the composition according to the present invention does not comprise bumetanide, or a pharmaceutically acceptable salt thereof. Bumetanide is a diuretic typically used in the treatment of heart failure and swelling. The composition according to the present invention does not contain any bumetanide i.e. 0 wt % bumetanide.

In an alternative embodiment, the present invention is directed to a composition comprising ibudilast, or a pharmaceutically acceptable salt thereof, for use in the treatment of Fragile X syndrome, wherein ibudilast is the only active agent in the composition. By only active agent it is meant that the composition does not contain other components which may be used in the treatment of Fragile X syndrome, in particular components that are capable of modulating intracellular calcium concentration. The composition may contain excipients such as fillers or binders.

In the present invention, the composition may be administered in a variety of dosage forms. In one embodiment, the composition may be formulated in a format suitable for oral, rectal, parenteral, intranasal or transdermal administration or administration by inhalation or by suppository.

The composition may be administered orally, for example as tablets, troches, lozenges, aqueous or oily suspensions, dispersible powders or granules. Preferably, the composition is formulated such that it is suitable for oral administration, for example tablets and capsules.

The composition may also be administered parenterally, whether subcutaneously, intravenously, intramuscularly, intrasternally, transdermally or by infusion techniques. Ibudilast may also be administered as suppositories.

The composition may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed.

The present invention also provides an inhalation device containing the composition of the present invention. Typically said device is a metered dose inhaler (MDI), which contains a pharmaceutically acceptable chemical propellant to push the medication out of the inhaler.

The composition may also be administered by intranasal administration. The nasal cavity's highly permeable tissue is very receptive to medication and absorbs it quickly and efficiently. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing the composition according to the present invention. The composition may also be administered by transdermal administration.

For topical delivery, transdermal and transmucosal patches, creams, ointments, jellies, solutions or suspensions may be employed. The present invention therefore also provides a transdermal patch containing the composition.

The composition may also be administered by sublingual administration. The present invention therefore also provides a sub-lingual tablet comprising the composition.

The composition may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In an embodiment of the invention, the composition is administered in an effective amount to treat the symptoms of Fragile X syndrome. An effective dose will be apparent to one skilled in the art, and is dependent on a number of factors including age, sex, weigh, which the medical practitioner will be capable of determining.

In a preferred embodiment, the composition comprises 10 mg to 300 mg of ibudilast, preferably 20 mg to 150 mg ibudilast.

The composition may be administered once a day, twice a day, three times a day or four times a day.

In an embodiment of the invention, the composition is administered at least once a day. Preferably it is administered as a single daily dose. Preferably the single daily dose comprises 10 mg to 300 mg ibudilast, preferably 20 mg to 150 mg of ibudilast.

In an embodiment of the invention, the composition is administered twice daily. Preferably each dose is 20 mg to 100 mg ibudilast, or 20 mg to 50 mg of ibudilast.

Preferably, the dosage regime is such that the total daily dosage of ibudilast does not exceed 300 mg.

In order to treat Fragile X syndrome, the composition comprising ibudilast is used in a chronic dosage regime i.e. chronic, long-term treatment.

The present invention also relates to use of ibudilast, or a pharmaceutically acceptable salt thereof, for the manufacture of a medicament for use in the treatment of Fragile X syndrome, wherein the medicament does not comprise sulindac or a pharmaceutically acceptable salt thereof, or bumetanide, or a pharmaceutically acceptable salt thereof. This embodiment of the invention may have any of the preferred features described above.

The present invention also relates to a method of treating Fragile X syndrome comprising administering the patient with a composition comprising ibudilast or a pharmaceutically acceptable salt thereof, wherein the composition does not comprise sulindac or a pharmaceutically acceptable salt thereof, or bumetanide, or a pharmaceutically acceptable salt thereof, wherein the patient is not being administered bumetanide. Preferably the patient is not being administered a substance capable of modulating intracellular calcium concentration as described above. This embodiment of the invention may have any of the preferred features described above. The method of administration may be according to any of the routes described above.

For the avoidance of doubt, the present invention also embraces prodrugs which react in vivo to give a compound of the present invention.

The following study illustrates the invention.

### Study 1

### Animals

Fmr1 knockout 2 (Fmr1 K02) mice were generated by deletion of the promoter and first exon of Fmr1. The Fmr1 KO2 it is both, protein and mRNA null. In this study we used Fmr1 KO2 and wild-type (WT) littermates generated on a C57BL/6J background and repeatedly backcrossed onto a C57BL/6J background for more than eight generations.

### Animal Housing

The Fmr1 KO2 mice were housed in 4-5 per cage groups of the same genotype in a temperature-(21±1° C.) and humidity-controlled room with a 12-hr light-dark cycle (lights on 7 a.m.-7 p.m.). Food and water were available ad libitum. Mice were housed in commercial plastic cages, and experiments were conducted in accordance with the requirements of the UK Animals (Scientific Procedures) Act, 1986. Protocols were reviewed and approved by the IEB, University of Chile Institute review board. All experiments were conducted with the staff blinded to genotype and drug treatment. Separate investigators prepared and coded the dosing solutions, allocated the mice to the study treatment groups, dosed the animals, and collected the behavioral data.

### Treatment Groups

There were four treatment groups per compound in the study with 10 male mice used per treatment group (all at 8 weeks of age): Group 1: wild-type littermate mice treated with vehicle (WT-Veh), Group 2: Fmr1 KO2 mice treated with vehicle (KO-Veh), Group 3: Fmr1 KO2 mice treated with 12 mg/kg ibudilast (KO-ibudilast).

### Behavioral Testing

For experiments, all mice were tested once in the same apparatus. Prior to testing, mice were placed in the apparatus for some minutes before the experiment. The apparatus was cleaned with moist and dry tissues before testing each mouse. The aim was to create a low but constant background mouse odor for all experimental subjects. Testers were blind to the genotype and treatment during all testing and data analysis. We assessed weight loss, fur loss, walking, eyes open, eye discharges and general behavior. All signs indicated that all treatments were well tolerated by the Fmr1KO2 mice and WT littermates at all times.

### Open Field (Hyperactivity)

The open-field apparatus was used to test hyperactivity. The apparatus was a gray PVC-enclosed arena 50×9×30 cm divided into a 10×10 cm grid. Mice were brought to the experimental room 5-20 min before testing. A mouse was placed into a corner square facing the corner and observed for 3 min. The number of squares entered by the whole body (locomotor activity) was counted. The movement of the mouse around the field was recorded with a video tracking device for 3 min (version NT4.0, Viewpoint).

### Nesting

The test was performed in individual cages. Normal bedding covered the floor to a depth of 0.5 cm. Each cage was supplied with a “Nestlet,” a 5 cm square of pressed cotton batting (Ancare). Mice were placed individually into the nesting cages 1 hr. before the dark phase, and the results were assessed the next morning. Nest building was scored on a 5 point scale.

Score 1: The Nestlet was largely untouched (>90% intact).
Score 2: The Nestlet was partially torn up (50-90% remaining intact).
Score 3: The Nestlet was mostly shredded but often there was no identifiable nest site: <50% of the Nestlet
Score 4: An identifiable, but flat nest <90% of the Nestlet was torn up, the material was gathered into a flat nest with walls higher than the mouse height curled up on its side) on less than 50% of its circumference.
Score 5: A (near) perfect nest: >90% of the Nestlet was torn up, the nest was a crater, with walls higher than mouse body height on more than 50% of its circumference.

### Fear Conditioning

The dependent measure used in contextual fear conditioning was a freezing response following a pairing of an unconditioned stimulus (foot shock), with a conditioned stimulus, a particular context. Freezing is a species-specific response to fear, which has been defined as “absence of movement except for respiration”. This may last for seconds to minutes depending on the strength of the aversive stimulus, the number of presentations, and the degree of learning achieved by the subject. Testing involved placing the animal in a novel environment (dark chamber), providing an aversive stimulus (a 1-sec electric shock, 0.2 mA, to the paws), and then removing it.

### Social Interaction

In the three-chambered sociability task, a subject mouse was evaluated for its exploration of a novel social stimulus (novel mouse). The three-chambered social approach task monitors direct social approach behaviors when a subject mouse is presented with the choice of spending time with either a novel mouse or an empty cup. Sociability is defined as the subject mouse spending more time in the chamber containing the mouse than in the empty chamber. Preference for social novelty is defined as spending more time in the chamber with the novel mouse. The apparatus is a rectangular three-chamber box, where each chamber measures 20 cm (length)×40.5 cm (width)×22 cm (height). Dividing walls are made from clear perplex, with small openings (10 cm width×5 cm height) that allow access into each chamber. The three chamber task was lit from below (10 lux). The mice were allowed to freely explore the three-chamber apparatus over three 10 min trials. During the trial one wire cup was placed upside down in one of the side chambers and a novel mouse was placed under another wire cup in the other side chamber (novel mouse stimulus), leaving the middle chamber empty. The location of the novel mouse across trials was counterbalanced to minimize any potential confound due to a preference for chamber location. The time spent exploring the novel mice was scored as exploration ratio.

### Statistical Analysis of Behavioral Data

Data were analyzed by one-way ANOVA with a Dunnett's multiple comparison post hoc test in which WT Vehicle group was used as reference and compared with all the other groups

Interpretation of the statistics:
NS: The means of WT group vs any other group are not significantly different therefore the treatment equalize the levels to WT (p>0.05)
WT group and any other group means are statistically different therefore the treatment is not enough to equalize to WT levels
   * P≤0.05
   ** P≤0.01
   *** P≤0.001
   **** P≤0.0001

### CONCLUSION

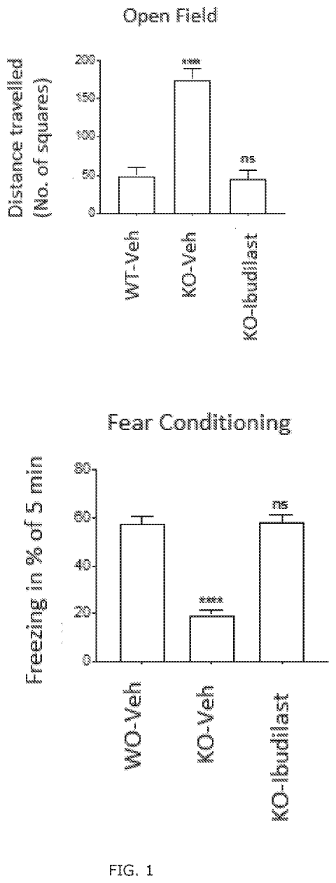

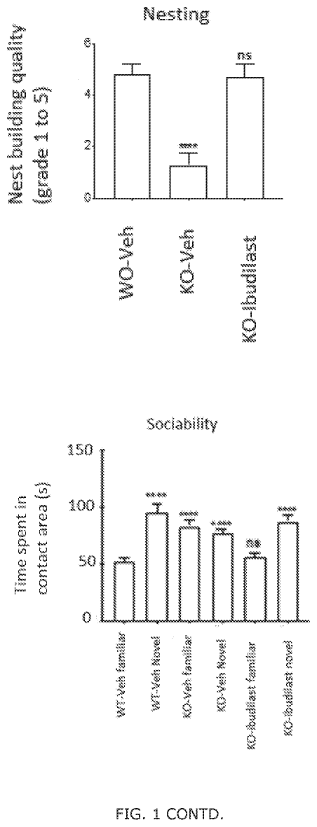

As can be seen from FIG. 1, treatment with a composition comprising ibudilast fully rescued open field, nesting, fear conditioning and sociability. This is evidence that a composition according to the present invention, comprising ibudilast, is successful in treating Fragile X syndrome.

1-14. (canceled)

15. A method of treating Fragile X syndrome comprising administering a composition comprising ibudilast, or a pharmaceutically acceptable salt thereof, to a patient, wherein the composition does not comprise sulindac, or a pharmaceutically acceptable salt thereof, or bumetanide, or a pharmaceutically acceptable salt thereof.

16. (canceled)

17. The method of claim 15, wherein the patient is human.

18. The method of claim 15, wherein the amount of ibudilast in the composition is selected from 10 mg-300 mg and 20 mg-150 mg.

19. The method of claim 15, wherein the composition is administered as a single daily dose.

20. The method of claim 19, wherein the amount of ibudilast in the single daily dose is selected from 10 mg-300 mg and 20 mg-150 mg.

21. The method of claim 15, wherein the composition is administered twice per day.

22. The method of claim 21, wherein the amount of ibudilast in each dose is independently selected from 20 mg-100 mg and 20 mg-50 mg.

23. The method of claim 15, wherein the composition is administered orally.

24. The method of claim 15, wherein the composition is administered parenterally, transdermally, sublingually, rectally or by inhalation.

25. The method of claim 15, wherein the patient is exhibiting signs of hyperactivity, social anxiety, memory loss, disruptive behaviour, or combinations thereof.

26. The method of claim 15, wherein the patient is exhibiting signs of hyperactivity, memory loss, disruptive behaviour, or combinations thereof.
